(19) United States
(12) Patent Application Publication
(10) Pub. No.: US 2003/0228369 A1
(43) Pub. Date: Dec. 11, 2003

(54) Process for conversion of high viscosity fluids and compositions thereof

(76) Inventor: Eric Hauser Kuhrts, Bodega, CA (US)

Correspondence Address: ERIC H. KUHRTS, PO BOX 16, BODEGA, CA, 94922, US

(21) Appl. No.: 10/140,495

(22) Filed: May 6, 2002

(51) Int. Cl. A61K 35/78; B29B 9/00; A61K 31/355; A61K 31/202; A61K 9/14

(52) U.S. Cl. 424/489; 424/725; 514/560; 514/458; 424/778; 424/729; 424/734; 264/5

(57) ABSTRACT

Disclosed is a process for producing powders from high viscosity fluids including mixing a mixture comprising a high viscosity fluid and at least one absorbing agent until a dry dispersion is produced. The process does not require the need for water or organic solvents, and is therefore non-aqueous in nature. Also disclosed are pharmaceutical compositions that include a high viscosity fluid, and at least one absorbing agent, wherein the percentage of high viscosity fluid in a finished dry powder is greater than 30%. In addition, there is disclosed a process for conversion of the alpha acids in hops extract to iso-alpha acids, and pharmaceutical compositions thereof.

### FIELD OF THE INVENTION

This invention relates to a non-aqueous process for manufacturing powders from high viscosity liquids, and compositions thereof. The process and compositions are particularly useful for producing pharmaceutical formulations from fluids or oils that would benefit from conversion to powders. The resulting powders may be used to manufacture standard solid dosage forms of therapeutic agents such as tablets, capsules, drink mix powders, candy bars or other confections and can be manufactured in a way to yield a high percentage of core material at a very economical cost. The process consists of converting a high viscosity fluid or oil such as a nutraceutical oil, vitamin, oleoresin, or botanical extract paste into a dry powder. No water or organic solvents are used in the process. The process is particularly useful for converting the oleoresin produced form the supercritical carbon dioxide extraction of the botanical humulus Lupulus L or hops, into a high yield powder. The process can also be used to convert the principle active components of the botanical resins in hops, the alpha acids, into iso-alpha acids. Compositions that are a result of the process are also included.

### BACKGROUND OF THE INVENTION

Many high viscosity fluids have useful utilitarian properties, but need to be converted into a fine free flowing powder so that they can then be employed in various applications. A free flowing powder can be weighed more precisely, and can be used in machinery to manufacture pharmaceutical dosage forms without clogging the apparatus or plugging various portals. In the pharmaceutical industry, many therapeutic agents are high viscosity fluids, and would need to be converted to a powder to enable a proper dosage form such as a tablet or a capsule. Many food ingredients, nutrients, cosmetics, and feed stuffs are also produced as high viscosity fluids, especially those substances that are in an oil base. Once a high viscosity fluid is converted into a powder, it can then be further processed into many different dosage forms such as tablets, two piece hard shell gelatin capsules, or powders that can be reconstituted in liquids or added to foods or confections.

It is an additional feature of this invention to produce powders from high viscosity fluids that are the result of organic solvent based extraction or supercritical carbon dioxide extraction. A preferred extraction would be supercritical carbon dioxide. One such example is the botanical extract hops (humulus Lupulus L.). The dried cones of the hops plant are extracted using supercritical carbon dioxide, which results in a higher yield of active therapeutic principles than a solvent based extract. Supercritical extraction also eliminates the possibility of solvent residues remaining in the extraction after evaporation of the liquid solvent. Supercritical carbon dioxide extraction concentrates the active components from botanicals, but the resulting product is usually a slurry or thick viscous fluid such as an oleoresin. This high viscosity fluid needs to be converted into a powder to be useful as a pharmaceutically acceptable dosage form. Because the powder produced from a viscous fluid that is the product of the supercritical carbon dioxide process, contains a higher percentage of active principles than a solvent based extract, there is a real practical need to turn this extract into an easy to formulate dosage form. This powder can then be used in tablets, capsules, drink mixes or confections without the limitations that are inherent to the fluid.

Pharmaceutical or therapeutic fluids are limited to incorporation into soft gelatin capsules, which require special machinery. Furthermore, not all liquids can be incorporated into softgel capsules. Liquids such as water, propylene glycol, glycerin and low molecular alcohols, ketones, acids, amines, and esters, cannot be files in softgel capsules unless they are at very low concentrations. Water at a concentration greater than 20% will dissolve the gelatin shell. The principle liquids that can filled in softgel capsules are primarily water immiscible liquids such as vegetable oils, aromatic oils, aromatic and aliphatic hydrocarbons, chlorinated hydrocarbons, ethers and esters, and including water miscible nonvolatile liquids, such as polyethylene glycols and nonionic surfactants.

In addition, the pH of a fill liquid for softgels is limited to above 2.5 and below 7.5. At low pH's, the gelatin is hydrolyzed and will cause the capsule to leak, whereas at higher pH's the gelatin is tanned, which results in decreased solubility of the shell. Even emulsions of oil and water are not suitable for encapsulation in softgels because they will eventually break up and release the water, which will dissolve the softgel. Therefore, there is a significant need to convert a high viscosity fluid into a powder so that it can be incorporated into a dosage form other than a softgel. Furthermore, there is also a need to convert the fluid into a powder with a high percentage of active principles or a large concentration of drug or other therapeutic agent in the powder.

Another feature of this invention is the production of therapeutic powders that can be manufactured into tablets without the need for further wet granulations. The term coined by the pharmaceutical manufacturing industry for this attribute is “direct compression”. A directly compressable powder does not need to be blended with many other excipients in an aqueous slurry (wet granulation) and then baked in ovens to dry. Once dry, the powder needs to be further milled into a powder because after the drying process, the material appears in a crust, cake, or flake like form. Milling is necessary to crush the granulation into a powder. In this invention, powders are produced in one step, and the powders so produced can be directly tableted without wet granulation.

The present invention is non-aqueous, and the high viscosity fluid is processed in a way to yield a high percentage of active component powder that is at least 50% or greater of the original slurry extract. Therefore, if a given plant extract has a level of a particular active principle of 10% in the form of a high viscosity fluid, the non-aqueous powder conversion described herein would result in a minimum yield of 5% of the active principle.

In general, pharmaceutical dosage forms are multi-particle formulations that when ingested in capsule form, rapidly disintegrate into a large number of subunits. This is suitable for drugs that are effective at relatively low doses, or dose levels that can fit into a capsule that is a reasonable size. The amount of drug that can fit into a two piece hard shell capsule that is easy for most people to swallow is at most about 800 mg. based on bulk density of the compound. But when large doses are required, such as for example with nutraceuticals, essential oils, or botanical substances, it is desirable to deliver the least number of dosage forms to enable good patience compliance. Ideally, this would be a single capsule or tablet. Patient compliance drops significantly when more than one dosage form is required for therapeutic effect, or when more than one dose is necessary per day. In addition, large doses of nutraceuticals or dietary supplements would be preferable taken in a powder dosage form that can be mixed with a liquid and consumed as a beverage in a single large dose.

There are many different ways to microencapsulate drugs. Many of these methods can be found in “Microcapsules and Microencapsulation Techniques”, 1976, M. H. Goucho, and Microcapsules and other Capsules, 1979, also by M. H. Goucho. Another resource book is “Aqueous Polymeric Coatings For Pharmaceutical Dosage Forms”, 1989, Marcel Dekker, Inc. Most of the methods of producing sustained-release microparticles can be classified into either physical or chemical systems. Physical methods would include such techniques as pan coating, gravity-flow, centrifuge, and the Wurster Process. The Wurster Process employs a high velocity air stream that is directed through a cylindrical fluid bed in which the particles are suspended in the air. A coating is sprayed onto the suspended particles, and the particles flow out the top of the cylinder and descend back to the layer of fluid. The flow of air-dries the coating, so that successive layers can be applied repeatedly by further spraying. Variables that control the process include the number of cycles, temperature, pressure, and humidity, and can be used to provide the desired coating composition and thickness.

Spray drying is one of the most common methods for creating powder from fluids. Spray drying has the limitation of low yields, and the high viscosity fluid must be solubulized or diluted to enable the fluid to be sprayed without clogging the spray nozzles. The resulting powders are usually below 20% of the original high viscosity fluid, and frequently, 5-10%, and therefore suffer from significant dilution. Spray drying is usually accomplished by dissolving maltodextrin in water and dissolving the therapeutic agent into the same solution and spray drying until a powder is produced, usually with about a 5% moisture level. Typically, a 30% solution of maltodextrin in water is made up to which the therapeutic agent is then dissolved. The solubility of the therapeutic agent is therefore an issue, and usually after spray drying, a 10% yield results. This means there was a 90% dilution. For example, spray drying of hops extracts in this manner, results in no greater than a 10% yield of the active principles or 10% of the starting level of alpha acids that were in the original extract. If one were to start with just the powdered leaves of the hops plant, instead of an organic solvent extract or a supercritical CO2 extract, one would have about a 12% level of alpha acids. Therefore, it is not practical to spray dry a botanical extract such as hops for pharmaceutical or therapeutic purposes, because there is a significant loss in potency.

Fluid bed granulation, agglomeration, or coating is also one of the most common techniques used at the present time for production of powders. Fluidized bed equipment is available as “top spray”, bottom spray”, and “tangential-spray”. The core drug is first preheated in the vessel to about 30° C. with hot air, placing the particles in suspension. The floating particles are then sprayed with an aqueous suspension to provide a coating, while drying at the same time. Inlet temperature, spray rate, and air throughput must be adjusted to provide optimum end product. Furthermore, the finished particles must be subjected to a post-drying period at around 40° C., where any residual moisture can be driven of. In some case, this last drying period may be up to 24 hours. In this case, the drug or therapeutic agent must already be in the form of a powder. Both traditional spray drying and fluid bed drying or agglomeration involve aqueous solutions or organic solvents. Fluid bed and spray drying are discussed in FLUID BED SPRAY DRYING OF A PROTEIN FORMULATION—A CASE STUDY, Rubino. O. Pharmaceutical Engineering, November 2000, which is incorporated herein as reference.

Many of the polymers that could be used to make powders in the fluid bed process require solvents such as acetone, isopropyl alcohol, chlorinated solvents, alkanes, methyl ethyl ketone, cyclohexane, toluene, carbon tetrachloride, chloroform, and the like. Evaporation of the solvents becomes an environmental concern, and in many states, it is illegal to release these emissions into the atmosphere. Aqueous or water based polymers are limited mainly to ethyl cellulose and methacrylic acid esters such as poly methacrylate dispersions. In addition, 10-20% of a suitable plasticizer such as triethyl citrate must be added to the polymer. For example, U.S. Pat. No. 5,603,957 uses a solvent-based polymer system to deliver aspirin over a 24-hour period. Preferred solvents are acetone/alkanol mixtures, or cyclohexane, toluene, or carbon tetrachloride. Castor oil, a low melting point oil, is also included in the polymer solvent mix.

Typical aqueous ethyl cellulose polymers currently in wide use include; Surelease®, Colorcon, West Point, Pa., and Aquacoat®, FMC Corporation, Philadelphia, Pa. In the Aquacoat® brochure available on their web site, it is recommended that for sustained-release applications, at least a two hour curing time at 60° C. be conducted to insure reproducible release profiles. This should be done in a tray dryer. Subjecting drugs and other therapeutic compounds such as botanical extracts to 60° C. temperatures for 2 hours or more is likely to result in a loss of potency or degradation of active principles, and is especially problematic for substances with low melting points. Botanical extracts, in particular, have many volatile compounds that can be destroyed if kept at high temperatures for long periods.

Another polymer in common use for pharmaceutical applications is Eudragit®, Huls America, Somerset N.J. This is a neutral methacrylic acid ester with a small proportion of trimethylammonioethyl methacrylate chloride. This polymer is also applied using the fluid bed process, or can be used in a standard wet granulation procedure.

Wet granulation involves mixing the drug or therapeutic agent with water in a conventional high-speed mixer until a pasty mass, and then dried in an oven over 24 hours at 60° C.

Wet granulations have the additional draw back in that they can effect the potency of botanical extracts by causing instability, or transformation. In addition, when dried at 60° C., many sensitive active principles are lost.

As mentioned above, spray drying high viscosity fluids on a maltodextrin carrier is the preferred method for converting wet substances to dry powders. This method is less than ideal in that the yields are usually very low, and the high viscosity fluid or paste must be diluted with polysorbate 80 or glucose to reduce the viscosity and enable it to be sprayed without clogging the nozzles of the spray apparatus. The ratio of ingredient to carrier such as maltodextrin is usually form 1:2 to 1:6. This means that if the ingredient to be spray dried or fluid bed spray dried is a protein, then the protein to maltodextrin ratio could be as high as 1 part protein to 6 parts maltodextrin.

Another method of producing powders is by starting with sugar spheres or nonpareils. The sugar serves as a seed for the creation of a particle. The sugar spheres are also processed in a fluid bed granulator, but the drug must be dissolved in a aqueous solution and sprayed onto the sugar spheres, followed by spray coating with polymers that produce sustained-release as previously mentioned. This system results in large particles that are not acceptable in most drink mix applications, and botanical extracts cannot be dissolved enough to use in this system. The therapeutic agent needs to be absorbed into the sugar particle. The smallest starting particle size for non-pareils is about 60 mesh (US standard sieve number). After coating, the particles are often 30 mesh and larger. The large particle size also presents a problem when encapsulating or tableting.

High intensity mixers such as the Littleford W-10 laboratory mixer, have about a 0.2 cubic foot working capacity, stainless steel construction, a 3 HP variable speed drive, and 45 PSIG heat transfer. The mixing unit is jacketed to enable passage of hot water or steam around the vessel to elevate the temperature of the internal contents if desired. The mixing blades inside the vessel are capable of high RPM, and are typically operated at 1-3,000 RPM. High intensity mixing produces fluidized bed mixing action, assuring absolute axial and radial mixing. High turnover mixing and high shear mixing are facilitated by high intensity mixers. Larger high intensity, high shear mixers are available with capacities that enable scale up to commercial production batches. An ideal intermediate size mixer of this type is the Littleford FM-130, which has a 130 liter (4.6 cubic feet), or 34 gallon capacity. Larger mixers of this type are available up to 25,000 liter capacity. Commercial batches of 1,000 kilos or more are possible in larger units.

The key feature of the mixer necessary to produce the invention described herein is that it be a high shear and high intensity mixer. Mixing technology has many types of subtlety, such as degrees of speed, precision, efficiency, configuration of mixing plows, turnover volume and shear. The Littleford mixers have a unique mixing action that is created by the mixing elements that produce intense, but gentle intermingling of the materials of the mix in a mechanically fluidized bed. In a high intensity mixer, the powder particles are fluidized (suspended in air) by mechanical motion. In a fluid bed granulator, the powder particles are actually suspended by high-pressurized air itself (air suspension). A fluid bed granulator blows the particles into the air and keeps them suspended, while aqueous fluids are sprayed onto the particles. The various configurations and locations of the mixing elements in a high intensity mixer like the Littleford FM-130, and the resulting mechanical motion are designed to force the product into appropriate components of axial and radial motion. This assures a quick and thorough mixing of the absorbent agent and the high viscosity fluid so as to enable a dry powder to be formed without the use of an aqueous solution such as water, or an organic solvent as is typically done in a wet granulation mixing or fluid bed granulation. Furthermore, the instant invention does not require the high viscosity fluid or the carrier (absorbent material) to be diluted in water or organic solvent, as is done in the prior art. The process described herein therefore differs substantially from spray drying or fluid bed spray drying or agglomeration.

### SUMMARY OF THE INVENTION

It is the object of the present invention to provide a high yield powder from a high viscosity fluid without the need of an additional aqueous fluid other than the high viscosity fluid itself. It is also the object of the present invention to provide a means of converting botanicals or pharmaceuticals that can be isomerized by heat to their respective isomers as part of the same process. It is a further objective of the present invention to provide a process for the conversion of hops extracted by supercritical carbon dioxide, into a high yield powder using the same process, and in that process, converting all or any desired percentage of the alpha acids in hops extract to iso-alpha acids. The entire process can be done without the need for water or organic solvents as is common to the prior art.

### DETAILED DESCRIPTION OF THE INVENTION

In accordance with the invention, there is provided a powder that is produced by mixing the high viscosity fluid with suitable absorbent carriers such as silica and maltodextrin in a jacketed mixing vessel, such as a Littleford high intensity mixer, to form a dry granulation. This process does not use water i.e., the maltodextrin is not dissolved in water as is done in spray drying. Once the granulation is formed, a dry powder is produced that is free flowing. An ideal candidate for this process is the thick viscous resin produced form the supercritical carbon dioxide extraction of hops. When this viscous resin is added to the high intensity mixing vessel with maltodextrin and silica, a dry granulation is formed without the need of an aqueous solution. Furthermore, the hops extract does not need to be dissolved in the aqueous solution and polysorbate 80, glycerin, or glucose are not needed as further diluents. When fully mixed, the temperature of the vessel can be increased to convert some of the active principles in hops (humulus Lupulis L) to isomers. In the case of hops, the principle therapeutic agents are known to be alpha acids. The alpha acids are converted to iso-alpha acids by continuing to process the powder conversion in the jacketed mixing vessel at an elevated temperature of about 150 degrees F. for about 15 minutes or more. This dry granulated/microencapsulated powder that has absorbed the high viscosity fluid is then suitable for tableting or filling into two piece hard shell capsules. The resulting powder may also be isomerized, which in the case of hops is the desirable form used in the production of beer, and is more soluble in the human gastrointestinal tract due to greater solubility.

The apparatus that is used to manufacture the powder can be a Littleford vertical or horizontal high intensity mixer (LittleforDay, Florence Ky.), or a comparable high intensity mixer or plow mixer that is jacketed with a hot water or steam bath. If the Littleford high intensity/high shear mixer is used, the heat produced by the jacked vessel, and the work input from the mixer itself can be used to convert the alpha acids in hops to iso-alpha acids. The heat produced in the jacket is produced by either steam or hot water that runs through the jacket. The unique mixing action of the auger shaft or plows (blades) revolving at a high rate of speed causes the particles to fluidize in free space, providing a high volume rate of material transfer throughout the entire length of the vessel. This results in the mixing, blending and adsorption of the high viscosity fluid onto the various absorbent carriers in a very efficient and economical fashion. In addition, the vessel can be fitted with high speed impact choppers to enhance mixing and or drying. After processing this way, the material is cooled and discharged as a free flowing powder. High intensity mixers like the Littleford mixers are not the same as blenders such as V-blenders or ribbon blenders.

The silica can be a high porosity spherical silica such as fumed silica with a high oil absorption capacity and small surface area, or various salts of silica. The average silica particle size distribution is usually about 0.01 to 0.05 microns as determined by electron microscope. The oil absorption of silica materials is about 200-500 ml/100 g, and the bulk specific gravity about 0.1 g/ml or less. The maltodextrin component can be any suitable maltodextrin or other suitable carrier that will dilute and absorb high viscosity liquids. Maltodextrin is produced from corn starch and is a complex carbohydrate. The combination of silica and maltodextrin is important, because maltodextrin alone is usually not sufficient to absorb a high viscosity fluid such as an oil or oleoresin. Other absorbant materials such as carbohydrates, proteinaceous materials such as sodium casienate, soy isolate, or whey protein, and fibers such as pectin, guar gum, or carboxymethylcellulose may be used.

### DETAILED DESCRIPTION OF THE INVENTION

The compositions of the invention are processed by mixing first, a high viscosity fluid, silica, and maltodextrin or other absorbent material in a jacketed high intensity mixer until a dry powder or dry granulation is formed, and discharging. The resulting powder is free flowing, dry, and of small particle size distribution, all desirable attributes for further manufacturing or handling for various utilitarian uses.

One of the preferred uses of such compositions is for pharmaceutical or therapeutic products and food additives. In particular, this method of production of powders is very useful for processing of botanical extracts and other nutraceutical oils or oleoresins. Some examples are fish oil and hops (Humulus lupulus L) extract. Fish oil is produced as an oily fluid with a characteristic fish like odor. Hops, like many other botanical extracts, is extracted as a paste or resin. If extracted by organic solvent method, a high viscosity paste is produced which must be dried (the solvent must be evaporated) and is usually placed onto a carrier. This is achieved by evaporation followed by spray drying. If supercritical CO2 extraction is used, the resulting extract is also a paste (high viscosity fluid), and while there is no need to evaporate off residue solvent, the thick paste is very difficult to use in solid dosage forms. The method and compositions described herein offer an excellent solution to the end products of botanical extraction, because this high viscosity paste can be converted to a relatively high yield powder. Furthermore, the subsequent microencapsulation helps to slow down degradation of sensitive principles in the extract, or helps to slow down oxidation of oils due to exposure to oxygen, moisture, or light. The resulting end product is therefore further stabilized by the complete process described herein.

Any suitable drug, therapeutic or prophylactic agent, nutraceutical, food additive, or botanical substance, cosmetic, fertilizer, or animal feed, that exists in a high viscosity form, such as an oil or oleoresin, or other such botanical paste, can be converted into a powder according to this process. A broad range of materials are therefore useful. Representative non-limiting examples would be; fish oil, omega 3 fatty acids, conjugated linoleic acid (CLA), docosahexaenoic acid (DHA), vitamin E, carotenoids such as beta carotene, tocotrienols, flax seed oil, hops (Humulus lupulus L), kava kava, saw palmetto, astaxanthin, lutein, lycopene, various fruit pastes, extract of spices such as rosemary, curcumin, and oregeno. Essentially any high viscosity fluid produced by supercritical CO2 extraction or other extraction methods that result in high viscosity fluids or slurrys that need to be dried or evaporated and converted into a useful powder can benefit from this process.

Useful dosage forms that can be made from such powders include, without limitation, oral forms such as tablets, capsules, beads, granules, aggregates, powders, gels, solids, semi-solids, or suspensions. Injectable forms, lotions, transdermal delivery systems including dermal patches, implantable forms or devices, aerosols or nasal mists, suppositories, salves and ointments are also useful. Cosmetic powders can also be produced.

The inventive compositions have great versatility in their application. The compositions can be used for wound management such as by direct application to bums, abrasions, skin diseases or infections and the like. Other uses such as packing agents for nasal wounds or other open wounds are also contemplated.

Additionally, antioxidants, preservatives, and essential oils may be incorporated into the matrix to add additional desirable smell or flavor characteristics, or to further stabilize compounds subject to oxidation.

Fertilizers, fungicides, cosmetics and food additives, would be non-drug applications for the process. Slow release of fertilizers and fungicides in the soil is especially desirable for nitrogen containing formulas.

Examples of classes of additives include excipients, lubricants, hydrocolloid suspending agents, buffering agents, disintegrating agents, stabilizers, foaming agents, pigments, coloring agents, fillers, bulking agents, sweetening agents, flavoring agents, fragrances, release modifiers, ect.

A variety of additives or carriers can be incorporated into the inventive compositions for their intended functions. These additives are usually used in small amounts, but if used as absorbing agents, they may be used in larger amounts. In some cases, additives such as hydrocolloids are used as suspending agents, as for example in a powdered drink mix that is reconstituted in liquid. Anti-oxidants or other preservatives may also be added.

Useful additives, some of which are absorbent agents, include, for example, gelatin, vegetable proteins such as sunflower protein, soybean proteins, cotton seed proteins, peanut proteins, rape seed proteins, blood proteins, egg proteins, acrylated proteins, casein, soy isolate protein, whey protein; water-soluble polysaccharides such as alginates, carrageenans, guar gum, agar-agar, gum arabic, and related gums (gum ghatti, gum karaya, gum tragacanth), pectin; cellulose, water-soluble derivatives of cellulose: alkylcelluloses, hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxpropylmethylceflulose, hydroxbutylmethylceflulose, cellulose esters and hydroxyalkylcellulose esters such as: cellulose acetate phthalate (CAP), carboxyalky I celluloses, carboxyalkylalkylcelluloses, carboxyalkylcellulose esters such as carboxymethyl cellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVP/vinyI acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylan-finoethyl group, which may be quaternized if desired; and other similar polymers.

Processing aids such as sucrose, polydextrose, maltodextrin, PEG 1500, polysorbate 80, lactose, maltose, and the like may also be used.

### STEP 1

The first step of the process is to convert the high viscosity fluid to a dry, small particle size, free flowing powder. This is done by adding the high viscosity fluid to a special type of mixer that is fitted with plows (blades) or augers that can rotate at high speed or RPM. One example of such a mixer is the Littleford W-10 high intensity mixer, which is jacketed, so the vessel can be heated with hot water or steam. The high viscosity fluid, carriers such as maltodextrin, and silica are added to the vessel according to the following weight percentages;

High viscosity fluid: 30-60%, maltodextrin: 40-70%, and silica: 2-10%. In most cases, the high viscosity fluid will be about 50%, the maltodextrin about 45%, and the silica about 5%. Salts of silica may be used. All of the components are mixed in the high intensity mixer at high RPMs, for 10 to 20 minutes until a dry granulation is achieved. This dry granulation consists of the high viscosity fluid absorbed onto the absorbent carrier. Not water or other aqueous solution or organic solvent is used. Choppers on the same unit can also be used to break up the particles if necessary, although this is rarely necessary.

### OPTIONAL STEP 2

After step one is performed, and without the need to transfer the powder to another vessel, the temperature of the vessel can be increase by circulating hot water or steam in the jacket to about 150 degrees F., while continuing to mix the contents for 20-60 minutes. The temperature of the unit is then lowered while mixing continues, and the resulting powder is then discharged, once room temperature is reached. The resulting powder is of small particle size, dry, and free flowing. This additional part of the process was used to convert essentially all or a part of the alpha acids in hops to iso-alpha acids. The isomerization of the alpha acids was accomplished without the need of chemicals such as potassium hydroxide or magnesium oxide as is typically done for the beer industry. The usual method for converting alpha acids in hops resin to iso-alpha acids is simply to boil the resin in a container that can be suspended in water. But suspending the resin in hot water in this fashion only results in about 50% of the alpha acids being converted to iso-alpha acids. The process described herein can result in virtually complete conversion of hops resin alpha acids to iso-alpha acids, in 1 hour or less.

### EXAMPLES

### Example 1

Fish oil is added to a high intensity, high shear, plow mixer (Littleford W-10) which was capable of operating at high temperatures because it was jacketed with a second layer to allow hot water to flow around the vessel. Silica (3%) and maltodextrin (47%) are added to the fish oil (50 weight percent). The fish oil, maltodextrin, and silica are blended at high speed. After complete mixing (about 15-20 minutes), the fish oil is converted into a free flowing, fine dry powder. A high speed chopper operating at 10 hp was fitted at the discharge point. The resulting granules were small and free flowing. The weight percent of the fish oil in the finished product was about 50%. The resulting fish oil powder had a reduced smell of fish, and was assimilated after ingestion without significant burping. Other essential oils such as rosemary oil can be added to the matrix during processing to provide a further smell masking if desired. It has been found that as little as ½% rosemary oil will impart additional desirable olfactory properties to the finished product. Vanilla extract also provides a pleasing overtone. According to the above process, these spices or essential oils can be dispersed in the oil matrix in a uniform way so as to remain in equal measure with the proportion originally sought.

### Example 2

Conjugated linoleic acid (CLA) oil was charged to a Littleford W-10 high shear mixer with a hot water jacket to allow circulating hot water to keep the vessel hot. 5% silica and 45% maltodextrin were added to the vessel and mixed thoroughly until a dry powder was produced. The work input was increased to 2000 RPM for 10 minutes, and then adjusted down to about 600 RPM for 5 minutes. After mixing and granulating the various components, absorption of the oil had occurred, resulting in a dry free flowing powder. The resulting particles were small, powder like, and free flowing.

### Example 3

Astaxanthin oleoresin is charged to a Littleford high intensity mixer with maltodextrin and silica sufficient to absorb the resin, and mixed at 1000 RPM. The RPMs are then decreased to maintain the power draw to within the allowable motor amperage. Unexpectedly, after 3-5 minutes the oil is fully absorbed and mixed with the carrier materials, and upon inspection, the batch is fully granulated. Astazanthin is a fat soluble nutrient, a xanthophyl pigment which is an oxygen derivative of the carotenoid family. It is a powerful antioxidant derived from microalgae.

Hops (Humulus lupulus) is extracted with supercritical CO2, and the resulting paste is standardized for alpha acids such as humulon. The resulting extract contains from about 40-80% alpha acids. Hops has been in use by the beer industry for hundreds of years. Usually, the alpha acids in hops are converted to iso-alpha acids through a process that involves heating the hops

[symbol]

[symbol]

Supercritical fluid technology is a more recent and superior means of extracting and concentrating the active principles that are contained in botanical extracts. Furthermore, supercritical fluid extraction is not a solvent based system, so it results in solvent free extractions, and is less harmful to the environment, because there is no need to evaporate the solvents. CO2 is the most commonly used material in supercritical fluid extraction and fractionation. Supercritical CO2 extraction also allows for better separation and fractionation of certain components in hops that may not be necessary for a particular application, such as the elimination of estrogenic components.

extract in solution with potassuin hydroxide. The alpha acids in hops pellets or dried hops flowers or leaves can be can be converted to iso-alpha acids by grinding the powder with magnesium oxide, and storing under elevated temperature (about 150 degrees F.) for about 72 hours. A typical hops powder consisting of dried leaves or flowers would contain about 10% alpha acids, and this would be converted to 10% iso-alpha acids in the process. The iso-alpha acids are the form preferred by the beer industry. More recently, hops has been shown to exhibit many interesting therapeutic properties primarily related to the alpha acids.

Extraction of hops yields various essential oils, oleoresins, and alpha and beta acids. The primary alpha acids contained in hops are humulon, cohumulone, hulupone, adhumulone, and xanthohumols. These alpha acids are not soluble at low pH. For example, the pH of gastric fluid is about 1.2, and at this pH, the alpha acids in hops such as humulon are not soluble. Even at the higher pH of the small intestine, which is 7.5, the alpha acids are only sparingly soluble. The bioavailablilty of the alpha acids in the gastrointenstinal tract, will be very low due to the low solubility, and this will effect the onset of therapeutic effect as well as the bioavailability. The alpha and beta acids in hops in their native form, or as extracted by either solvent based or supercritical carbon dioxide, will exhibit very low bioavailability in-vivo.

The primary beta acids in hops are lupulone, colupulone, and adlupulone. Hops resin is obtained from the yellow vesicles in the flowers of the hops plant. Extraction of hops resin is usually done using accepted extraction techniques with such solvents as hexane or ethyl alcohol, which concentrates the alpha and beta acids. Liquid carbon dioxide under super critical conditions, or chromatography can be used to separate the alpha and beta fractions.

Solvent based extracts of hops can yield about 5-15% alpha acids, while supercritical carbon dioxide extracts yield much higher concentrations of alpha acids, usually from 40-90% alpha acids. Therefore, even after powder conversion using the process described in the present invention, the resulting powders produced form supercritical extracts are more potent than powders produced from organic solvent extracts or powdered hops cones. As will be seen from the example given below, hops powders consisting of 30% total alpha acids can be produced from a starting high viscosity fluid produced by supercritical CO2 extraction with 60% alpha acids content. Furthermore, a 30% iso-alpha acid containing powder can be produced form the same 60% starting extract if desired, or a 50:50 mixture of 50% alpha and 50% iso-alpha acids can be produced. All of the alpha acids can be converted to iso-alpha acids, or part of the alpha acids can be converted to iso-alpha acids.

One of the discoveries of this invention is directed to a composition that results in more soluble and bioavailable formulations of hops by converting the alpha acids to iso-alpha acids, preferably the alpha acid humulon to iso-humulon. The iso-alpha acids are therefore more effective, for example, for pain relief from inflammation such as osteoarthritis, or trauma induced pain, due to better bioavailability and faster onset of action The major iso-alpha acids are trans-isocohumulone, trans-isohumulone and trans-isoadhumulone. There are also tetrahydroiso-alpha acids, hexahydroiso-alpha acids, p-iso-alpha acids.

Normally, the alpha acids in hops extract are isomerized by heating the high viscosity extract with potassium hydroxide or another mineral salt in aqueous solution. The resulting hops extract yields primarily iso-alpha acids, and this is the method used for production if iso-alpha acids used by the beer industry. As mentioned before, simply heating hops extract in boiling water in a suitable vessel only results in about 50% conversion to iso-alpha acids.

Why are iso-alpha acids beneficial? At pH 2 or below, the solubility of the alpha acids in hops is essentially zero. At pH 3-4 the alpha acids are only sparingly soluble, for example, a solution of only 100 ppm is possible at a pH of 4. At pH 6, only a 1-2% solution can be made, and at pH 10 about a 10% solution is possible. As mentioned before, the beta acids are virtually insoluble at low pH. However, iso-alpha acid is much more soluble at low pH as well as high pH. For example at pH 7.5 a 20% aqueous solution can be made of iso-alpha acid, whereas only a 10% solution can be made of alpha acid. A 30% aqueous solution can be made by incorporation of potassium hydroxide in heated distilled water to bring the pH up to 9. The iso-alpha acids are therefore at least 100% more soluble and available at the pH of the human small intestine, and even more soluble at the pH of the stomach, which is about 1.2. Neither the alpha acids or the beta acids are soluble at the pH of the stomach. Thus, the iso-alpha acids will exhibit greater absorption and faster onset of action because they will become available for absorption early on, because their dissolution will start to occur in the stomach and continue as they move into the small intestine. This will result in better availability in the proximal small intestine, and throughout the mid and distal small intestine, where most drugs are absorbed.

While hops extract has many desirable properties, the widespread use of a potent extract such as is produced by supercritical extraction, wherein certain active principles have been concentrated, has been hindered due to the nature of thick viscous paste. A supercritical CO2 extract of hops was therefore subjected to the following process;

### Example 4

Supercritical CO2 extract of Hops paste containing 60% alpha acids was added to a jacketed high intensity mixer with 5% silica and 45% maltodextrin. The mixing plows are started and mix the contents under high shear and speed with significant turn over until the high viscosity fluid is dried into a powder by admixture with the carriers. This process occurs in about 10-20 minutes and the resulting powder discharged. The hops was a faint greenish yellow, free flowing, very fine powder that could now be tableted or placed into two piece hard shell capsules. Analysis by HPLC gave the following profile;

Analysis Results:

| | | | HPLC, DCHA |
|---|---|---|---|
| | HPLC, ICE-2 Standard | | Iso-alpha standard |
| Sample | % Alpha acids | % Beta Acids | % Iso-alpha acids |
| Methanol | 18.9 | 8.7 | 11.4 |
| extraction | | | |

As can be seen from the analysis, slightly more than ⅓ of the alpha acids were converted to iso-alpha acids in this process, and the resulting powder yielded a total of 30.30% alpha acids, or approximately 50% of the starting material. This is a much higher percentage of alpha acids than either the powdered hops cones or an organic solvent based extract, or a supercritical CO2 extract that has been spray dried. In fact, the resulting powder in this example is at least twice as potent in terms of alpha acid content than any other powder produced by any process currently known by the inventor to be in the prior art. As mentioned previously, powdered leaves or cones (flowers), or organic solvent extracts yield only 5-15% alpha acids versus the 30% achieved by the invention.

### Example 5

A 60% total alpha acid resin produced by extraction of hops cones with supercritical CO2 was obtained as a thick viscous paste. The resin was added to the vessel of a high intensity mixer (Littleford M-60) with maltodextrin and silica and converted to a powder while the temperature of the vessel was elevated to 150 degrees F. for approximately 60 minutes. The resulting powder was analyzed by HPLC according to American Society of Brewing Chemists. Report of Subcommittee on alpha-acids and beta acids in Hops and Hop Extracts by HPLC. Journal 48:138-140, 1990, and yielded the following;

Analysis Results:

| | | | HPLC, DCHA |
|---|---|---|---|
| | HPLC, ICE-2 Standard | | Iso-alpha standard |
| Sample | % Alpha acids | % Beta Acids | % Iso-alpha acids |
| Methanol | 0.0 | 8 | 30 |
| extraction | | | |

In example 2 above it can be seen that essentially all of the alpha acids were converted to iso-alpha acids.

While the present invention is described above in connection with the preferred or illustrative embodiments, those embodiments are not intended to be exhaustive or limiting of the invention, but rather, the invention is intended to cover any alternatives, modifications or equivalents that may be included within its scope as defined by the appended claims.

What is claimed is:

1. A non-aqueous process for producing dry powders from high viscosity fluids comprising: dispersing a high viscosity fluid in a mixer capable of high intensity mixing, with one or more suitable absorbent carriers, and mixing until a dry powder is produced.

2. The process of claim 1 wherein the high viscosity fluid is selected from; oils, oleoresins, botanical extracts, fruit extracts, spices, vitamins; pharmaceuticals, fungicides, and fertilizers.

3. The process of claim 1 wherein the carrier is a carbohydrate, a proteinaceous material, fibers, or silica.

4. The process of claim 3 wherein the percentage of carrier used is from 10% to 80% by weight in the finished powder.

5. The process of claim 1 wherein the range of carrier is about 20% to 60%% by weight in the finished powder.

6. The process of claim 1 wherein the range of carrier is about 50% by weight in the finished powder.

7. The process of claim 1 wherein a high shear/high intensity mixer such as a Littleford mixer with a heating jacket is employed.

8. The process of claim 2 wherein the high viscosity fluid is fish oil, omega 3 fatty acids, conjugated linoleic acid, docosahexaenoic acid, eicosapentaenioc acid, vitamin E, flax seed oil, carotenoids, tocotrienols, astaxanthin, lutein, lycopene, essential oils, hops, kava kava, saw palmetto, spice oils, and mixtures thereof.

9. A dry pharmaceutical powder composition comprising; a) a high viscosity fluid, b) an absorbing agent, wherein the high viscosity fluid is greater than 30% by weight of the dry powder.

10. The composition of claim 9 wherein the high viscosity fluid is hops extract.

11. The composition of claim 10 wherein the hops is extracted using supercritical carbon dioxide.

12. The composition of claim 11 wherein the supercritical carbon dioxide extract of hops contains at least 20-90% alpha acids.

13. The composition of claim 12 wherein the supercritical carbon dioxide of hops contains 40-60% alpha acids.

14. The composition of claim 9 wherein the dry pharmaceutical powder is hops extract containing greater than 15% alpha acids.

15. The composition of claim 9 wherein the high viscosity fluids are; fish oil, conjugated linoleic acid, essential oils, tocotrienols, lutein, lycopene, EPA, DHA, flax seed oil, hops (Humulus lupulus), kava kava, saw palmetto, and astaxanthin.

16. The composition of claim 9 wherein at least one absorbing agent comprises: carbohydrates, proteinaceous materials, fibers, silica or a combination thereof.

17. The composition of claim 16 wherein the carbohydrates comprise: maltodextrin, corn starch, corn syrup solids, and glucose.

18. The composition of claim 16 wherein the proteinaceous materials comprise sodium casseinate, casein, soy protein isolate, or whey protein.

19. The composition of claim 16, wherein at least one of the fibers comprise acacia gum, guar gum, cellulose, carboxymethylcellulose, or pectin.

20. The composition of claim 9 wherein the absorbing agents are maltodextrin and silica or a salt of silica.

21. The composition of claim 20 wherein the maltodextrin is 35 to 60% of the weight of the finished powder and the silica is 2 to 10%.

22. The composition of claim 20 wherein the maltodextrin is 40-55% and the silica is 3-5%.

23. The composition of claim 9 wherein the high viscosity fluid is about 45-60% of the dry powder.

24. The composition of claim 10 wherein the hops extract is about 50% and the absorbing agents are maltodextrin and silica.

25. A process for converting alpha acids in a hops extract to a dry powder comprising iso-alpha acids by mixing hops extract in a jacketed high intensity mixer with an absorbing agent at a temperature sufficient to convert some or all of the alpha acids to iso-alpha acids, and resulting in a dry free flowing powder.

26. A dry powder pharmaceutical composition comprising iso-alpha acids from hops extract, and an absorbing agent, wherein the iso-alpha acids are present in at least 10% by weight of the powder.

27. The pharmaceutical composition of claim 26 wherein the iso-alpha acids are 30 to 45% by weight of the powder.

28. The composition of claim 26 wherein the absorbing agents are maltodextrin and silica.

29. The composition of claim 26 wherein the hops extract is derived by supercritical carbon dioxide extraction.

30. The process of claim 25 wherein the hops extract is derived by supercritical carbon dioxide.
